(19) United States
(12) Patent Application Publication
(10) Pub. No.: US 2019/0069957 A1
(43) Pub. Date: Mar. 7, 2019

(54) SURGICAL RECOGNITION SYSTEM

(71) Applicant: Verily Life Sciences LLC, Mountain View, CA (US)

(72) Inventors: Joëlle K. Barral, Mountain View, CA (US); Ali Shoeb, Mill Valley, CA (US); Daniele Piponi, Oakland, CA (US); Martin Habbecke, Palo Alto, CA (US)

(21) Appl. No.: 15/697,189

(22) Filed: Sep. 6, 2017

(51) Int. Cl. A61B 34/20; G06T 7/00; G06K 9/32

(52) U.S. Cl. CPC A61B 34/20; G06T 7/0012; G06T 7/75; G06T 2207/30004; G06K 9/3233

(57) ABSTRACT

A system for robotic surgery includes a surgical robot with one or more arms, where at least some of the arms in the one or more arms holds a surgical instrument. An image sensor is coupled to capture a video of a surgery performed by the surgical robot, and a display is coupled to receive an annotated video of the surgery. A processing apparatus is coupled to the surgical robot, the image sensor, and the display. The processing apparatus includes logic that when executed by the processing apparatus causes the processing apparatus to perform operations including identifying anatomical features in the video using a machine learning algorithm, and generating the annotated video. The anatomical features from the video are accentuated in the annotated video. The processing apparatus also outputs the annotated video to the display in real time.

### TECHNICAL FIELD

This disclosure relates generally to systems for performing surgery, and in particular but not exclusively, relates to robotic surgery.

### BACKGROUND INFORMATION

Robotic or computer assisted surgery uses robotic systems to aid in surgical procedures. Robotic surgery was developed as a way to overcome limitations (e.g., spatial constraints associated with a surgeon's hands, inherent shakiness of human movements, and inconsistency in human work product, etc.) of pre-existing surgical procedures. In recent years, the field has advanced greatly to limit the size of incisions, and reduce patient recovery time.

In the case of open surgery, robotically controlled instruments may replace traditional tools to perform surgical motions. Feedback controlled motions may allow for smoother surgical steps than those performed by humans. For example, using a surgical robot for a step such as rib spreading, may result in less damage to the patients tissue than if the step were performed by a surgeon's hand. Additionally, surgical robots can reduce the amount of time in the operating room by requiring fewer steps to complete a procedure.

However, robotic surgery may be relatively expensive, and suffer from limitations associated with conventional surgery. For example, a surgeon may need to spend lots of time training on a robotic system before performing surgery. Additionally, surgeons may become disoriented when performing robotic surgery, which may result in harm to the patient.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. The drawings are not necessarily to scale, emphasis instead being placed upon illustrating the principles being described.

FIG. 1A illustrates a system for robotic surgery, in accordance with an embodiment of the disclosure.

FIG. 1B illustrates a controller for a surgical robot, in accordance with an embodiment of the disclosure.

FIG. 2 illustrates a system for recognition of anatomical features while performing surgery, in accordance with an embodiment of the disclosure.

FIG. 3 illustrates a method of annotating anatomical features encountered in a surgical procedure, in accordance with an embodiment of the disclosure.

### DETAILED DESCRIPTION

Embodiments of an apparatus and method for recognition of anatomical features during surgery are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The instant disclosure provides for a system and method to recognize organs and other anatomical structures in the body while performing surgery. Surgical skill is made of dexterity and judgment. Arguably, dexterity comes from innate abilities and practice. Judgment comes from common sense and experience. Exquisite knowledge of surgical anatomy distinguishes excellent surgeons from average ones. The learning curve to become a surgeon is long: the duration of residency and fellowship often approaches ten years. When learning a new surgical skill, a similar long learning curve is seen, and proficiency only obtained after performing 50 to 300 cases. This is true for robotic surgery as well, where co-morbidities, conversion to open procedure, estimated blood loss, procedure duration, and the like, are worse for inexperienced surgeons than for experienced ones. Surgeons are expected to see about 500 cases a year which span a variety of procedures. Accordingly, a surgeon's intrinsic knowledge of anatomy with respect to any one type of surgical procedure is inherently limited. The systems and methods disclosed here solves this problem using a computerized device to bring the knowledge gained from many similar cases to each operation. The system achieves this goal by producing an annotated video feed, or other alerts (e.g., sounds, lights, etc.) that inform the surgeon which parts of the body he/she is looking at (e.g., highlighting blood vessels in the video feed to prevent the surgeon from accidentally cutting through them). Previously, knowledge of this type could only be gained by trial and error (potentially fatal in the surgical context), extensive study, and observation. The system disclosed here provides computer/robot-aided guidance to a surgeon in a manner that cannot be achieved through human instruction or study alone. In some embodiments, the system can tell the difference between two structures that the human eye cannot distinguish between (e.g., because the structures' color and shape are similar).

The instant disclosure trains a machine learning model (e.g., a deep learning model) to recognize specific anatomical structures within surgical videos, and highlight these structures. For example, in cholecystectomy (removal of gallbladder), the systems disclosed here trains a model on frames extracted from laparoscopic videos (which may, or may not, be robotically assisted) where structures of interest (liver, gallbladder, omentum, etc.) have been highlighted. Once image classification has been learned by the algorithm, the device may use a sliding window approach to find the relevant structures in videos and highlight them, for example by delineating them with a bounding box. In some embodiments, a distinctive color or a label can then be added to the annotation. More generally, the deep learning model can receive any number of video inputs from different types of cameras (e.g. RGB cameras, IR cameras, molecular cameras, spectroscopic inputs, etc.) and then proceed to not only highlight the organ of interest, but also sub-segment the highlighted organ into diseased vs. non-diseased tissue, for example. More specifically the deep learning model described may work on image frames. Objects are identified within videos using the models previously learned by the machine learning algorithm in conjunction with a sliding window approach or other way to compute a similarity metric (for which it can also use a priori information regarding respective sizes). Another approach is to use machine learning to directly learn to delineate, or segment, specific anatomy within the video, in which case the deep learning model completes the entire job.

The system disclosed here can self-update as more data is gathered: in other words the system can keep learning. The system can also capture anatomical variations or other expected differences based on complementary information, as available (e.g., BMI, patient history, genomics, preoperative imagery, etc.). While learning currently requires lots of computational power, the model once trained can run locally on any regular computer or mobile device, in real time. In addition, the highlighted structures can be provided to the people who need them, and only when they need them. For example, the operating surgeon might be an experienced surgeon and not need visual cues, while observers (e.g., those watching the case in the operating room, those watching remotely in real time, or those watching the video at a later lime) might benefit from an annotated view. Solving the problem in this manner makes use of all the data available. The model(s) can also be retrained as needed (e.g., either because new information about how to segment a specific patient population becomes available, or because a new way to perform a procedure is agreed upon in the medical community). While deep learning is a likely way to train the model, many alternative machine learning algorithms may be employed such as supervised and unsupervised algorithms. Such algorithms include support vector machines (SVM), k-means, etc.

There are a number of ways to annotate the data. For example recognized anatomical features could be circled by a dashed or continuous line, or the annotation could be directly superimposed on the structures without specific segmentation. Doing so would alleviate the possibility of imperfections in the segmentation that could bother the surgeon and/or bare risk. Alternatively or additionally, the annotations could be available in a caption, or a bounding box could follow the anatomical features in a video sequence over time. The annotations could be toggled on/off by the surgeon, at will, and the surgeon could also specify which type of annotations are desired (e.g., highlight blood vessels but not organs). A user interface (e.g., keyboard, mouse, microphone, etc.) could be provided to the surgeon to input additional annotations. Note that an online version can also be implemented, where automatic annotation is performed on a library of videos for future retrieval and learning.

The systems and methods disclosed here also have the ability to perform real-time video segmentation and annotation during a surgical case. It is important to distinguish between spatial segmentation where, for example, anatomical structures are marked (e.g., liver, gallbladder, cystic duct, cystic artery, etc.) and temporal segmentation where the steps of the procedures are indicated (e.g., suture placed in the fundus, peritoneum incised, gallbladder dissected, etc.).

For spatial segmentation, both single-task and multi-task neural networks could be trained to learn the anatomy. In other words, all the anatomy could be learned at once, or specific structures could be learned one by one. For temporal segmentation, convolutional neural networks and hidden Markov models could be used to learn the current state of the surgical procedure. Similarly, convolutional neural networks and long short-term memory or dynamic time warping may also be used.

For spatial segmentation, the anatomy could be learned frame by frame from the videos, and then the 2D representations would be stitched together to form a 3D model, and physical constraints could be imposed to increase the accuracy (e.g., maximum deformation physically possible between two consecutive frames). Alternatively, learning could happen in 3D, where the videos—or parts of the videos, using a sliding window approach or Kalman filtering—would be provided directly as inputs to the model.

For learning, the models can also combine information from the videos with other a priori knowledge and sensor information (e.g., biological atlases, preoperative imaging, haptics, hyperspectral imaging, telemetry, and the like). Additional constraints could be provided when running the models (e.g., actual hand motion from telemetry). Note that dedicated hardware could be used to run the models quickly and segment the videos in real time, with minimal latency.

Another aspect of this disclosure consists of the reverse system: instead of displaying to the surgeon anatomical overlays when there is high confidence, the model could alert the surgeon when the model itself is confused. For example when there is an anatomical area that does not make sense because it is too large, too diseased, or too damaged for the device to verify its identity, the model could alert the surgeon. The alert can be a mark on the user interface, or an audio message, or both. The surgeon then has to either provide an explanation (e.g., a label) or he/she can call a more experienced surgeon (or a team of surgeons, so that inter variability is assessed and consensus labeling is obtained) to make sure he/she is performing the surgery appropriately. The label can be provided by the surgeon either on the user interface (e.g., by clicking on the correct answer if multiple choices are provided) or labels can be provided by audio labeling (“OK robot, this is a nerve”), or the like. In this embodiment, the device addresses an issue that often surgeons don't recognize: that the surgeon is misoriented during the operation—unfortunately surgeons often don't realize this error until they've made a mistake.

Heat maps could be used to convey to the surgeon the level of confidence of the algorithm, and margins could be added (e.g., to delineate nerves). The information itself could be presented as an overlay (e.g., using a semi-transparent mask) or it could be toggled using a foot pedal (similar to the way fluorescence imaging is often displayed to surgeons).

No-contact zones could be visually represented on the image, or imposed on the surgeon through haptic feedback that prevents (e.g., make it hard or stop entirely) the instruments from going in the forbidden regions. Alternatively, sound feedback could be provided to the surgeon when he/she approaches a forbidden region (e.g., the system beeps when the surgeon is entering a forbidden zone). Surgeons would have the option to turn on/off the real-time video interpretation engine at any time during the procedure, or have it run in the background but not display anything.

In the temporal embodiment, where surgical steps are learned and sequence prediction is enabled, whenever the model knows with high confidence what the next steps should be, these could be displayed to the surgeon, (e.g., using a semi-transparent overlay or haptic feedback that guides the surgeon's hand in the expected direction). Alternatively, feedback could be provided when the surgeon deviates too much from the expected path. Similarly, the surgeon could also ask the robot what the surgical field is supposed to look like a minute from now, be provided that information, and then continue the surgery without any visual encumbrance on the surgical field.

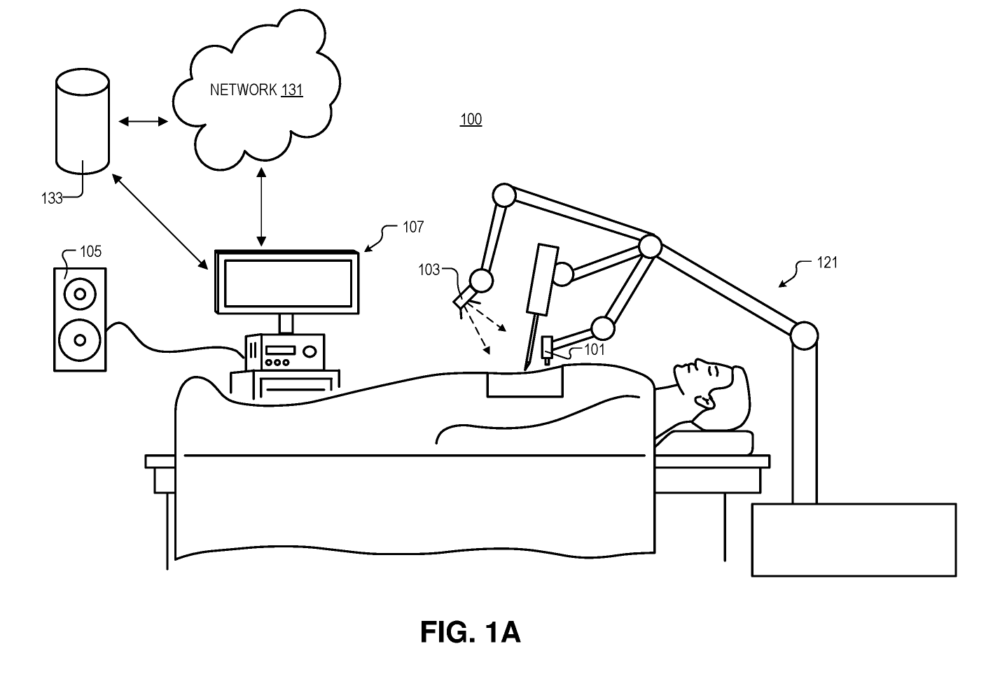

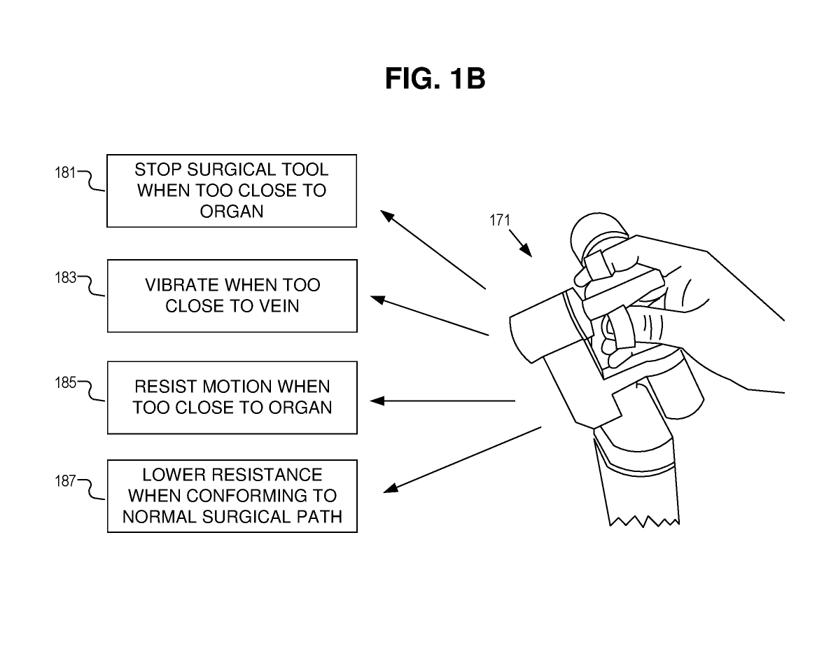

The following disclosure describes illustrations (e.g., FIGS. 1-3) of some of the embodiments discussed above, and some embodiments not yet discussed.

FIG. 1A illustrates system 100 for robotic surgery, in accordance with an embodiment of the disclosure. System 100 includes surgical robot 121, camera 101, light source, 103, speaker 105, processing apparatus 107 (including a display), network 131, and storage 133. As shown, surgical robot 121 may be used to hold surgical instruments (e.g., each arm holds an instrument at the distal ends of the arm) and perform surgery, diagnose disease, take biopsies, or conduct any other procedure a doctor could perform. Surgical instruments may include scalpels, forceps, cameras (e.g., camera 101) or the like. While surgical robot 121 only has three arms, one skilled in the art will appreciate that surgical robot 121 is merely a cartoon illustration, and that surgical robot 121 can take any number of shapes depending on the type of surgery needed to be performed and other requirements. Surgical robot 121 may be coupled to processing apparatus 107, network 131, and/or storage 133 either by wires or wirelessly. Furthermore, surgical robot 121 may be coupled (wirelessly or by wires) to a user input/controller (e.g., controller 171 depicted in FIG. 1B) to receive instructions from a surgeon or doctor. The controller, and user of the controller, may be located very close to the surgical robot 121 and patient (e.g., in the same room) or may be located many miles apart. Thus surgical robot 121 may be used to perform surgery where a specialist is many miles away from the patient, and instructions from the surgeon are sent over the internet or secure network (e.g., network 131). Alternatively, the surgeon may be local and may simply prefer using surgical robot 121 because it can better access a portion of the body than the hand of the surgeon could.

As shown, an image sensor (in camera 101) is coupled to capture a video of a surgery performed by surgical robot 121, and a display (attached to processing apparatus 107) is coupled to receive an annotated video of the surgery. Processing apparatus 107 is coupled to (a) surgical robot 121 to control the motion of the one or more arms, (b) the image sensor to receive the video from the image sensor, and (c) the display. Processing apparatus 107 includes logic that when executed by processing apparatus 107 causes processing apparatus 107 to perform a variety of operations. For instance, processing apparatus 107 may identify anatomical features in the video using a machine learning algorithm, and generate an annotated video where the anatomical features from the video are accentuated (e.g., by modifying the color of the anatomical features, surrounding the anatomical feature with a line, or labeling the anatomical features with characters). The processing apparatus may then output the annotated video to the display in real time (e.g., the annotated video is displayed at substantially the same rate as the video is captured, with only minor delay between the capture and display). In some embodiments, processing apparatus 107 may identify diseased portions (e.g., tumor, lesions, etc.) and healthy portions (e.g., an organ that looks “normal” relative to a set of established standards) of anatomical features, and generate the annotated video where at least one of the diseased portions or the healthy portions are accentuated in the annotated video. This may help guide the surgeon to remove only the diseased or damaged tissue (or remove the tissue with a specific margin). Conversely, when processing apparatus 107 fails to identify the anatomical features to a threshold degree of certainty (e.g., 95% agreement with the model for a particular organ), processing apparatus 107 may similarly accentuate the anatomical features that have not been identified to the threshold degree of certainty. For example, processing apparatus 107 may label a section in the video “lung tissue; 77% confident”.

As described above, in some embodiments the machine learning algorithm includes at least one of a deep learning algorithm, support vector machines (SVM), k-means clustering, or the like. Moreover, the machine learning algorithm may identify the anatomical features by at least one of luminance, chrominance, shape, or location in the body (e.g., relative to other organs, markers, etc.), among other characteristics. Further, processing apparatus 107 may identify anatomical features in the video using sliding window analysis. In some embodiments, processing apparatus 107 stores at least some image frames from the video in memory to recursively train the machine learning algorithm. Thus, surgical robot 121 brings a greater depth of knowledge and additional confidence to each new surgery.

In the depicted embodiment, speaker 105 is coupled to processing apparatus 107, and processing apparatus 107 outputs audio data to speaker 105 in response to identifying anatomical features in the video (e.g., calling out the organs shown in the video). In the depicted embodiment, surgical robot 121 also includes light source 103 to emit light and illuminate the surgical area. As shown, light source 103 is coupled to processing apparatus 107, and processing apparatus may vary at least one of an intensity of the light emitted, a wavelength of the light emitted, or a duty ratio of the light source. In some embodiments, the light source may emit visible light, IR light, UV light, or the like. Moreover, depending on the light emitted from light source 103, camera 101 may be able to discern specific anatomical features. For example, a contrast agent that binds to tumors and fluoresces under UV or IR light may be injected into the patient. Camera 103 could record the fluorescent portion of the image, and processing apparatus 107 may identify that portion as a tumor.

In one embodiment, image/optical sensors (e.g., camera 101), pressure sensors (stress, strain, etc.) and the like are all used to control surgical robot 121 and ensure accurate motions and applications of pressure. Furthermore, these sensors may provide information to a processor (which may be included in surgical robot 121, processing apparatus 107, or other device) which uses a feedback loop to continually adjust the location, force, etc. applied by surgical robot 121. In some embodiments, sensors in the arms of surgical robot 121 may be used to determine the position of the arms relative to organs and other anatomical features. For example, surgical robot may store and record coordinates of the instruments at the end of the arms, and these coordinates may be used in conjunction with video feed to determine the location of the arms and anatomical features. It is appreciated that there is a number of different ways (e.g., from images, mechanically, time-of-flight laser systems, etc.) to calculate distances between components in system 100 and any of these may be used to determine location, in accordance with the teachings of present disclosure.

FIG. 1B illustrates a controller 171 for robotic surgery, in accordance with an embodiment of the disclosure. Controller 171 may be used in connection with surgical robot 121 in FIG. 1A. It is appreciated that controller 171 is just one example of a controller for a surgical robot and that other designs may be used in accordance with the teachings of the present disclosure.

In the depicted embodiment, controller 171 may provide a number of haptic feedback signals to the surgeon in response to the processing apparatus detecting anatomical structures in the video feed. For example, a haptic feedback signal may be provided to the surgeon through controller 171 when surgical instruments disposed on the arms of the surgical robot come within a threshold distance of the anatomical features. For example, the surgical instruments could be moving very close to a vein or artery so the controller lightly vibrates to alert the surgeon (181). Alternatively, controller 171 may simply not let the surgeon get within a threshold distance of a critical organ (183), or force the surgeon to manually override the stop. Similarly, controller 171 may gradually resist the surgeon coming too close to a critical organ or other anatomical structure (185), or controller 171 may lower the resistance when the surgeon is conforming to a typical surgical path (187).

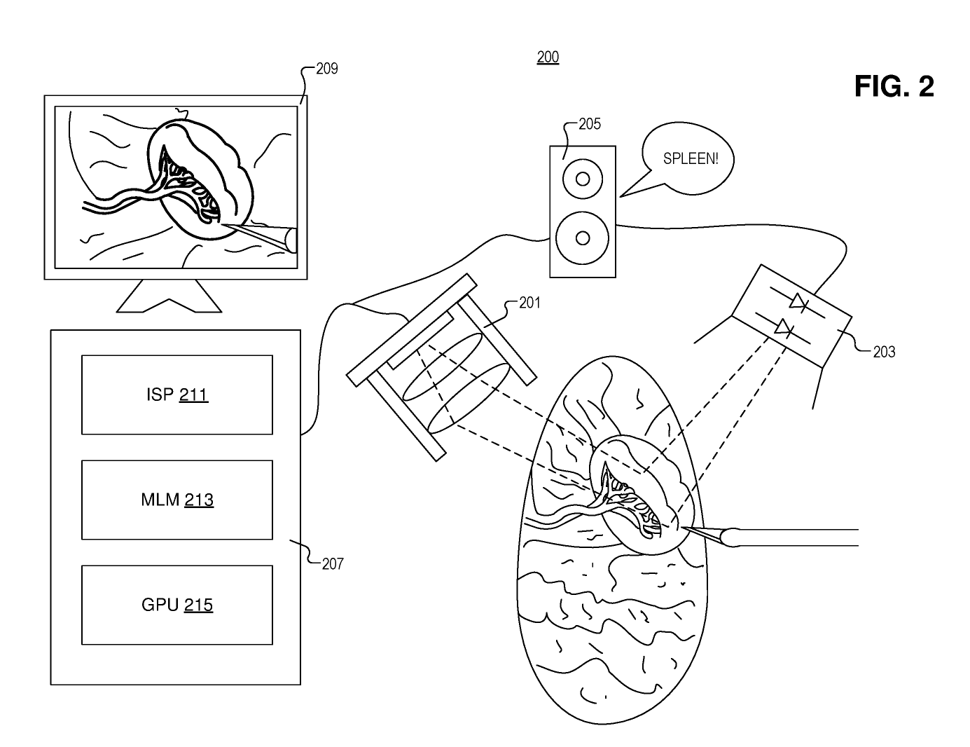

FIG. 2 illustrates a system 200 for recognition of anatomical features while performing surgery, in accordance with an embodiment of the disclosure. The system 200 depicted in FIG. 2 may be more generalized than the system of robotic surgery depicted in FIG. 1A. This system may be compatible with manually performed surgery, where the surgeon is partially or fully reliant on the augmented reality shown on display 209, or with surgery performed with an endoscope. For example, some of the components (e.g., camera 201) shown in FIG. 2 may be disposed in an endoscope.

As shown, system 200 includes camera 201 (including an image sensor, lens barrel, and lenses), light source 203 (e.g., a plurality of light emitting diodes, laser diodes, an incandescent bulb, or the like), speaker 205 (e.g., desktop speaker, headphones, or the like), processing apparatus 207 (including image signal processor 211, machine learning module 213, and graphics processing unit 215), and display 209. As illustrated, light source 203 is illuminating a surgical operation, and camera 201 is filming the operation. A spleen is visible in the incision, and a scalpel is approaching the spleen. Processing apparatus 207 has recognized the spleen in the incision and has accentuated (bolded its outline either in black and white or color) the spleen in the annotated video stream. In this embodiment, when the surgeon looks at the video stream the spleen and associated veins and arteries are highlighted so the surgeon doesn't mistakenly cut into them. Additionally, speaker 205 is stating that the scalpel is near the spleen in response to instructions from processing apparatus 207.

It is appreciated that the components in processing apparatus 207 are not the only components that may be used to construct system 200, and that the components (e.g., computer chips) may be custom made or off-the-shelf. For example, image signal processor 211 may be integrated into the camera. Further, machine learning module 213 may be a general purpose processor running a machine learning algorithm or may be a specialty processor specifically optimized for deep learning algorithms. Similarly, graphics processing unit 215 (e.g., used to generate the augmented video) may be custom built for the system.

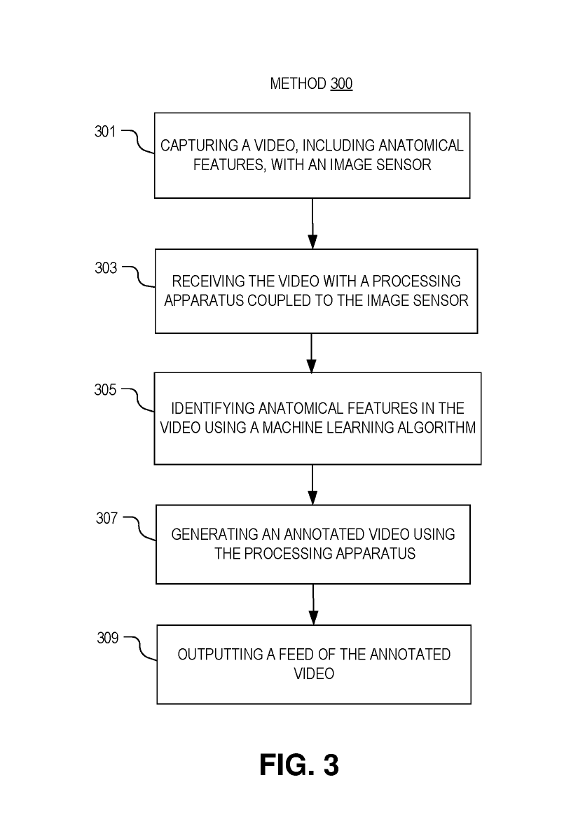

FIG. 3 illustrates a method 300 of annotating anatomical features encountered in a surgical procedure, in accordance with an embodiment of the disclosure. One of ordinary skill in the art having the benefit of the present disclosure will appreciate that the order of blocks (301-309) in method 300 may occur in any order or even in parallel. Moreover, blocks may be added to, or removed from, method 300 in accordance with the teachings of the present disclosure.

Block 301 shows capturing a video, including anatomical features, with an image sensor. In some embodiments, the anatomical features in the video feed are from a surgery performed by a surgical robot, and the surgical robot includes the image sensor.

Block 303 illustrates receiving the video with a processing apparatus coupled to the image sensor. In some embodiments, the processing apparatus is also disposed in the surgical robot. However, in other embodiments the system includes discrete parts (e.g., a camera plugged into a laptop computer).

Block 305 describes identifying anatomical features in the video using a machine learning algorithm stored in a memory in the processing apparatus. Identifying anatomical features may be achieved using sliding window analysis to find points of interest in the images. In other words, a rectangular or square region of fixed height and width scans/slides across an image, and applies an image classifier in order to determine if the window includes an interesting object. The specific anatomical features may be identified using at least one of a deep learning algorithm, support vector machines (SVM), k-means clustering, or other machine learning algorithm. These algorithms may identify anatomical features by at least one of luminance, chrominance, shape, location, or other characteristic. For example, the machine learning algorithm may be trained with anatomical maps of the human body, other surgical videos, images of anatomy, or the like, and use these inputs to change the state of artificial neurons. Thus, the deep learning model will produce a different output based on the input and activation of the artificial neurons.

Block 307 shows generating an annotated video using the processing apparatus, where the anatomical features from the video are accentuated in the annotated video. In one embodiment, generating an annotated video includes at least one of modifying the color of the anatomical features, surrounding the anatomical features with a line, or labeling the anatomical features with characters.

Block 309 illustrates outputting a feed of the annotated video. In some embodiments, a visual feedback signal is provided in the annotated video. For example, when surgical instruments disposed on arms of a surgical robot come within a threshold distance of the anatomical features, the video may display a warning sign, or change the intensity/brightness of the anatomy depending on how close to it the robot is. The warning sign may be a flashing light, text, etc. In some embodiments, the system may also output an audio feedback signal (e.g., where the volume is proportional to distance) to a surgeon with a speaker if the surgical instruments get too close to an organ or structure of importance.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise. Processes may also occur locally or across distributed systems (e.g., multiple servers).

A tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A system for robotic surgery, comprising:
a surgical robot with one or more arms, wherein at least some of the arms in the one or more arms holds a surgical instrument;
an image sensor coupled to capture a video of a surgery performed by the surgical robot;
a display coupled to receive an annotated video of the surgery; and
a processing apparatus coupled to the surgical robot to control the motion of the one or more arms, coupled to the image sensor to receive the video, and coupled to the display to supply the display with the annotated video, wherein the processing apparatus includes logic that when executed by the processing apparatus causes the processing apparatus to perform operations including:
identifying anatomical features in the video using a machine learning algorithm; and
generating the annotated video, wherein the anatomical features from the video are accentuated in the annotated video; and
outputting the annotated video to the display in real time.

2. The system for robotic surgery of claim 1, wherein the machine learning algorithm includes at least one of a deep learning algorithm, support vector machines (SVM), or k-means clustering.

3. The system for robotic surgery of claim 1, wherein the machine learning algorithm identifies the anatomical features by at least one of luminance, chrominance, shape, or location in the body.

4. The system for robotic of claim 1, wherein accentuating the anatomical features in the video includes at least one of modifying the color of the anatomical features, surrounding the anatomical feature with a line, or labeling the anatomical features with characters.

5. The system for robotic surgery of claim 1, further comprising a speaker coupled to the processing apparatus, wherein the processing apparatus further includes logic that when executed by the processing apparatus causes the processing apparatus to perform operations including:
outputting audio data to the speaker in response to identifying anatomical features in the video.

6. The system for robotic surgery of claim 1, wherein the processing apparatus further includes logic that when executed by the processing apparatus causes the processing apparatus to perform operations including:
identifying diseased portions of the anatomical features, and identifying healthy portions of the anatomical features; and
generating the annotated video, wherein at least one of the diseased portions or the healthy portion are accentuated in the annotated video.

7. The system for robotic surgery of claim 1, wherein the processing apparatus further includes logic that when executed by the processing apparatus causes the processing apparatus to perform operations including:
failing to identify other anatomical features to a threshold degree of certainty; and
generating the annotated video, wherein other anatomical features that have not been identified to the threshold degree of certainty are accentuated in the annotated video.

8. The system for robotic surgery of claim 1, further comprising a light source coupled to the processing apparatus, wherein the processing apparatus further includes logic that when executed by the processing apparatus causes the processing apparatus to perform operations including:
controlling the light source to emit light and vary at least one of an intensity of the light emitted, a wavelength of the light emitted, or a duty ratio of the light source.

9. The system for robotic surgery of claim 1, wherein the processing apparatus further includes logic that when executed by the processing apparatus causes the processing apparatus to perform operations including:
storing at least some image frames from the video in memory to train the machine learning algorithm.

10. The system for robotic surgery of claim 1, wherein identifying anatomical features in the video includes using sliding window analysis.

11. A method of annotating anatomical features encountered in a surgical procedure, comprising:
capturing a video, including anatomical features, with an image sensor;
receiving the video with a processing apparatus coupled to the image sensor;
identifying anatomical features in the video using a machine learning algorithm stored in a memory in the processing apparatus;
generating an annotated video using the processing apparatus, wherein the anatomical features from the video are accentuated in the annotated video; and
outputting a feed of the annotated video in real time.

12. The method of claim 11, further comprising performing the surgical procedure with a surgical robot, wherein the image sensor and the processing apparatus are included in the surgical robot.

13. The method off claim 12, further comprising providing a haptic feedback signal to a surgeon using the surgical robot when surgical instruments disposed on arms of the surgical robot come within a threshold distance of the anatomical features.

14. The method off claim 12, further comprising providing a visual feedback signal to a surgeon when surgical instruments disposed on arms of the surgical robot come within a threshold distance of the anatomical features, and wherein the visual feedback is provided on a display coupled to the processing apparatus to receive the feed of the annotated video.

15. The method of claim 12, further comprising outputting an audio feedback signal to a surgeon with a speaker coupled to the processing apparatus when surgical instruments disposed on arms of the surgical robot come within a threshold distance of the anatomical features.

16. The method of claim 11, further comprising illuminating the anatomical features with a light source coupled to the processing apparatus, wherein the processing apparatus causes the light source to emit light and vary at least one of an intensity of the light emitted, a wavelength of the light emitted, or a duty ratio of the light source.

17. The method of claim 11, wherein identifying anatomical features in the video using a machine learning algorithm includes using at least one of a deep learning algorithm, support vector machines (SVM), or k-means clustering.

18. The method of claim 17, wherein the machine learning algorithm identifies the anatomical features by at least one of luminance, chrominance, shape, or location in the body.

19. The method of claim 11, wherein generating an annotated video includes at least one of modifying the color of the anatomical features, surrounding the anatomical feature with a line, or labeling the anatomical features with characters.

20. The method of claim 11, further comprising training the machine learning algorithm to recognize the anatomical features using the video.

21. The method of claim 11, further comprising training the machine learning algorithm to recognize the anatomical features using at least one of images of the anatomical features, a second video of a previously recorded surgical procedure, or maps of a human body.

22. The method of claim 11, wherein identifying the anatomical features in the video includes using sliding window analysis to identify the anatomical features in each frame of the video.
